(19) United States
(12) Patent Application Publication
(10) Pub. No.: US 2015/0157209 A1
(43) Pub. Date: Jun. 11, 2015

(54) BIOMEDICAL DETECTION APPARATUS

(71) Applicant: BOARD OF TRUSTEES OF MICHIGAN STATE UNIVERSITY, East Lansing, MI (US)

(72) Inventor: Marcos Dantus, Okemos, MI (US)

(73) Assignee: BOARD OF TRUSTEES OF MICHIGAN STATE UNIVERSITY, East Lansing, MI (US)

(21) Appl. No.: 14/413,372

(22) PCT Filed: Jul. 3, 2013
(86) PCT No.: PCT/US2013/049214

§ 371 Date: Jan. 7, 2015

Related U.S. Application Data
(60) Provisional application No. 61/669,953, filed on Jul. 10, 2012.

(51) Int. Cl. A61B 5/00

(52) U.S. Cl. CPC A61B 5/0071; A61B 5/0086; A61B 5/7282; A61B 5/7415; A61B 5/725; A61B 5/742; A61B 5/4836; A61B 5/0075; A61B 5/0013; A61B 2576/00

(57) ABSTRACT

A biomedical apparatus employing laser light is provided. In another aspect, laser light is unfocused when it is emitted upon in vivo or exposed internal tissue during surgery. A further aspect provides a visual and/or audio warning to the surgeon during surgery if a cancer cell is detected, within one minute and more preferably within five seconds, of emission of laser light upon the targeted tissue.

### CROSS-REFERENCE TO RELATED APPLICATIONS

This application claims the benefit of U.S. Provisional Application No. 61/669,953, filed on Jul. 10, 2012. The entire disclosure of the above application is incorporated herein by reference.

### BACKGROUND

The present disclosure generally pertains to a biomedical system and more specifically to a biomedical diagnostic and treatment apparatus using a laser.

Various methods are known for detecting cancer cells within a margin area of healthy tissue adjacent to a removed tumor. For example, U.S. Pat. No. 8,043,603 entitled “Folate Targeted Enhanced Tumor and Folate Receptor Positive Tissue Optical Imaging Technology” which issued to Kennedy et al. on Oct. 25, 2011, discloses a laser light focused through a microscope which causes fluorescence captured by a camera and spectrograph. Furthermore, P. Bordenave et al., “Wide-Field Optical Coherence Tomography: Imaging of Biological Tissues,” Applied Optics, Vol. 41, No. 10, at 2059 (Apr. 1, 2002) discloses the use of optical coherence tomography employing interferometric imaging. In another example, U.S. Pat. No. 4,930,516 entitled “Method for Detecting Cancerous Tissue Using Visible Native Luminescence” which issued to Alfano et al. on Jun. 5, 1990, employs in vivo fluorescent spectrography with a focused laser beam. U.S. Pat. No. 7,505,811 entitled “Method and Apparatus for Examining Tissue for Predefined Target Cells, Particularly Cancerous Cells, and a Probe Useful in such Method and Apparatus” which issued to Hashimshony on Mar. 17, 2009, discloses an electro-optical probe using laser pulses plus electrical impedance measuring, and is sold as the Marginprobe® brand device. The disadvantages of all of these approaches are discussed in the Background section of U.S. Pat. No. 6,671,540 entitled “Methods and Systems for Detecting Abnormal Tissue Using Spectroscopic Techniques” which issued to Hochman on Dec. 30, 2003. Moreover, the lack of depth and speed of these conventional methods are discussed in the Background section of U.S. Pat. No. 7,372,985 entitled “Systems and Methods for Volumetric Tissue Scanning Microscopy” which issued to So et al. on May 13, 2008. All of the patents referenced hereinabove are incorporated by reference.

### SUMMARY

In accordance with the present invention, a biomedical apparatus employing laser light is provided. In another aspect, laser light is unfocused when it is emitted upon in vivo or exposed internal tissue during surgery. A further aspect provides a visual and/or audio warning to the surgeon during surgery if a cancer cell is detected, within one minute and more preferably within one second, of emission of laser light upon the targeted tissue. In yet another aspect, unfocused laser light, a detector and a programmed control system allow for cancer cell detection within at least a 10 mm³ volume of tissue during surgery, without interferometry, mapping or other time-consuming calculations involving determining the location of the cancer cell in the tissue. Computer software and a method of detecting the presence of undesirable cells in vivo, are additionally provided. In yet another aspect, the laser is used to kill virus, bacteria or cancer cells within at least a 10 mm³ volume of tissue through multiphoton excitation.

The present biomedical detection apparatus and procedure are advantageous over traditional devices and methods. For example, the present apparatus and method are extremely fast thereby allowing for essentially real time and almost instantaneous feedback to the surgeon during the surgical procedure; this avoids the current need for repeated surgeries separated by hours if not days, while slow image mapping is occurring. Moreover, the present apparatus and method are capable of detecting an undesirable or cancerous cell within a larger area and volume of tissue inside the patient during surgery faster than can otherwise be achieved with conventional devices. Additionally, the patient is not exposed to harmful ultraviolet light or radiation as are employed with some traditional systems. Further benefits and advantages will be seen from the following description and claims, taken in conjunction with the appended drawings.

### BRIEF DESCRIPTION OF THE DRAWINGS

FIG. 1 is a diagrammatic view showing a first embodiment of a biomedical detection apparatus of the present invention;

FIG. 2 is an enlarged diagrammatic view showing a first embodiment of the present apparatus;

FIG. 3 is an enlarged diagrammatic view showing a second embodiment of the present apparatus;

FIG. 4 is a diagrammatic cross-sectional view through a cancerous group of cells in a margin area of tissue;

FIG. 5 is a diagrammatic cross-sectional view showing cancerous cells totally surrounded within healthy tissue;

FIG. 6 is a flow chart showing programmable computer software employed in the present apparatus;

FIG. 7 is a diagrammatic illustration showing an alternating chirp embodiment of the present apparatus;

FIG. 8 is a flow chart showing programmable computer software employed in another embodiment of the present apparatus;

FIG. 9 is a perspective view showing another embodiment of the present apparatus;

FIG. 10 is a perspective view showing another embodiment of the present apparatus; and

FIGS. 11 and 12 illustrate self-contained wand embodiments of the present invention.

### DETAILED DESCRIPTION

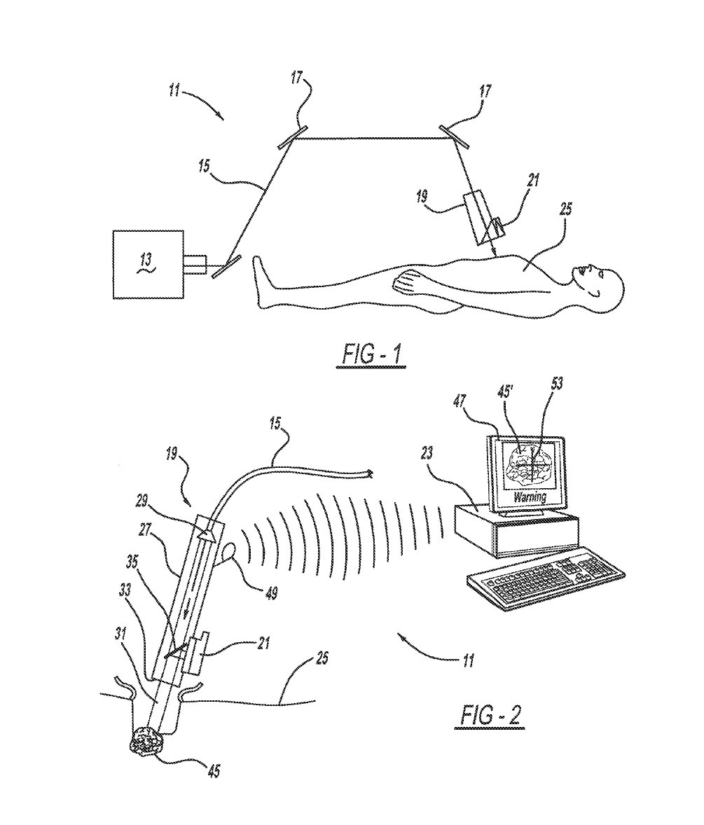

A first preferred embodiment of a biomedical detection apparatus 11 is shown in FIGS. 1 and 2. Apparatus 11 includes a laser 13, one or more optical fibers 15, reflective mirrors 17, a hand-held wand 19, a detector 21 and an electronic controller 23. These components can be packaged within one or more housings and protective conduits adjacent a gurney supporting a patient 25 in a hospital.

Laser 13 is preferably of a titanium sapphire, ytterbium, erbium, or chromium fosterite variety which is capable of emitting ultra-fast laser beam light pulses each having a duration of 100 femtoseconds or less, more preferably 50 fs or less, more preferably 30 fs or less, even more preferably 10 fs or less, and most preferably between 5-10 fs. The faster and shorter the pulse duration, the better since ten times shorter pulses require ten times less energy to induce the same amount of fluorescence. For example, at least 10¹⁰ W/cm² of peak power density is desired for two-photon excitation. Shorter pulses are advantageous because the less energy needed per pulse, the lower the possibility of thermally induced damage for the tissue of patient 25. A less expensive laser can also be employed if less pulse energy is required. Moreover, multiple ultra-fast laser pulses of 10 femtoseconds or less allow for improved averaging during each exposure frame for detector 21. It is further desirable to employ a MIIPS® pulse shaping system which can be obtained from Biophotonic Solutions, Inc. to characterize and compensate for spectral phase distortions in the laser pulses. It is preferred that optical fibers 15 are hollow to deter non-linear optical distortions in the pulses.

A. Detection of Rogue Cancer Cells During Open Surgery:

Wand 19 has an elongated and generally tubular housing 27 which is intended to be hand-held by the surgeon adjacent to patient 25. A collimator optic 29 is connected to fiber 15 inside of housing 27 for collimating and emitting laser pulses 31 from a distal end 33 of housing 27. A one-way reflective beam splitter 35 is also located within housing 27. Splitter 35 allows the emitted pulse 31 to pass therethrough but fluorescence light 37 emitted back from the tissue is then reflected at an offset angle to detector 21. The embodiment showed in FIG. 3 illustrates a reversed configuration where incoming laser light 31 is perpendicularly offset from the elongated direction of housing 27 of wand 19 while detector 21 is linearly aligned with housing 27. One or more focusing lenses 41 and a color filter 43 are present to receive and act upon the reflected fluorescing light between splitter 35 and detector 21.

Detector 21 is preferably a CCD camera which can provide a real-time digitized video display of targeted tissue 45 on a visual output display screen 47 of computer controller 23. Filter 43 will emphasize one or more fluorescing colors, green for example for a cancer cell, which contrast to the pink healthy tissue. Detector 21 communicates with computer controller 23 via an antenna 49 sending radio waves therebetween, although light or even hard wired communications can be used.

It is noteworthy that the laser light pulses 31 emitted from wand 27 are not focused. Nevertheless, it should be appreciated that a focusing lens may be employed in laser 13 upstream or before wand 27, and lens 41 is employed for the detector 21 through which only reflected or fluorescing light passes. Therefore, when the term “unfocused” is used with regard to the emitted laser beam light pulses for the present application, it should be appreciated that it means that the laser light emitted from wand 19 at the patient tissue 45 is not focused with any optical lens. The use of unfocused 30 fs laser pulses or even shorter pulses has the ancillary benefit of reducing the risk that the wand may blind the surgeon or nearby assistants if it is accidentally shined in their eyes; the eye disperses the unfocused laser pulses given the fast pulse durations. It should be appreciated that the unfocused emitted light of the present application is in stark contrast to microscopy, which by definition, requires an objective lens to focus the emitted laser pulses passing therethrough. Moreover, an objective disadvantageously illuminates a very small volume, measured in microns.

Wand 27 is used by a surgeon in real-time during a surgical procedure on patient 25. After the surgeon has removed the known cancerous or other undesirable cells from the patient, a fluorescent chemical marker is injected into the surgical area adjacent to the known cancerous cells. Alternately, the marker may be injected before or during the initial cancer removal step. A cancer-specific antibody conjugated to a fluorescent molecule protein or quantum dot would be suitable as the marker or tag. The surgeon thereafter holds the wand 27 immediately above or adjacent to the surrounding tissue 45 in vivo within the incision, in other words, exposing the internal tissue of the patient to emitted laser pulses 31. The surgeon slowly moves wand 27 in a back and forth zig/zag, spiral or other pattern adjacent to a tissue area of the removed, known cancerous cells; the fluorescent marker will cause any unknown, remaining cancerous cells to emit back fluorescent light 37 of a different color than that of the healthy tissue 45 for sensing and detection by camera 21. Again, filter 43 is matched to the marker so as to emphasize the cancer cell color.

Computer software, stored and used in non-transient memory of computer controller 23, operates the laser and receives the signal from the camera 21. Such memory includes and is not limited to RAM, ROM, removable memory, hard disc drives, and the like. The software optimizes, calibrates, and tests the laser system, such as by matching a filter to a marker. Then the software instruction cause the laser shutter to open so as to transmit each laser pulse to the wand, and thus, the patient tissue. Exposure and gain of the camera detector are automatically controlled by additional programmed instructions based on comparisons of actual data to pre-stored memory valves. Computer controller 23 further includes an output display screen 47 and an input keyboard or switches.

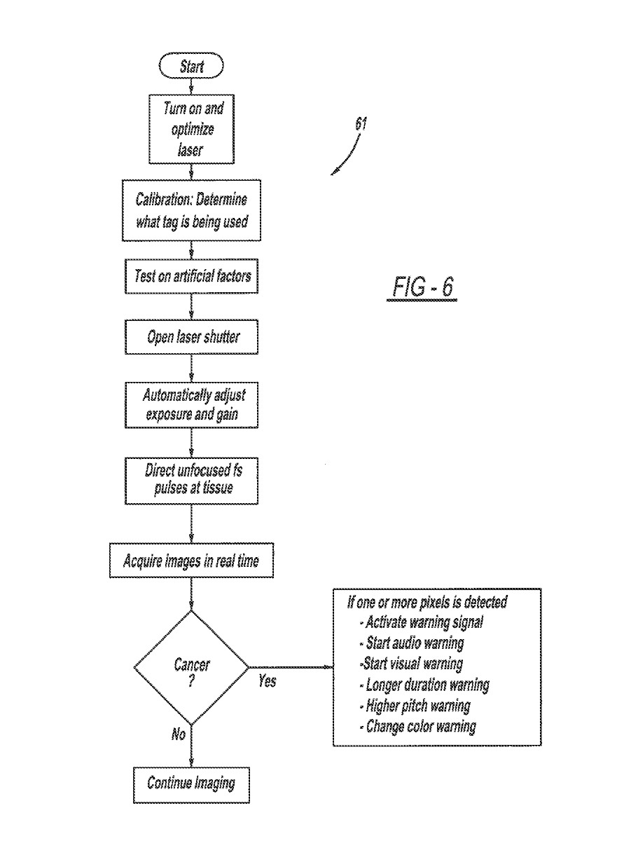

Computer software 61 includes multiple sets of programmed instructions or modules such as shown in FIG. 6. Software 61 includes instructions operably causing emission of unfocused laser light onto in vivo tissue during the surgery. It also includes instructions operably receiving signals corresponding to detected wide field imaging data fluoresced from a cancer cell in the in vivo tissue. Instructions also compare the detected data to stored values in memory. Furthermore, instructions operably send a notification signal, such as a warning sound or visual warning display on output screen 47, if the software determines that a cancer or undesirable cell is present. Such an audio warning may increase or change the pitch of the sound as the wand targets the emitted light closer to the cancer cell; a similar color or text change may also occur for the visual warning displayed.

It is noteworthy that the software instructions determine the presence of the cancer cell but without determining or calculating a location of the cancer cell. This is highly advantageous since the surgeon (which includes other nearby medical personnel) can be promptly warned of unknown remaining cancerous cells in an essentially real-time manner of less than five minutes, and more preferably less than one second, from when the surgeon targets a tissue area (as is shown by crosshairs 53, bull's-eyes, or other indicia on output screen 47). Such detection and essentially instantaneous reporting results allows the surgeon to conduct this process while the surgical area of the patient is still open and exposed, thereby preventing the trauma and time when metastasis can occur associated with conventional repeated surgeries separated by hours or days while awaiting three-dimensional image mapping results.

The software instructions and hardware are suitable for determining if cancer cell 51 is present within at least 10 mm² area, more preferably a 10 mm³ volume, and most preferably a 300 mm³ volume associated with dimension d (see FIG. 3) of the in vivo tissue 45 during surgery, and without interferometry. In an optional additional procedure, software instructions will allow the surgeon to vary at least one subsequent laser pulse, such as by increasing the energy thereof or changing a pulse shape, to activate an injected chemical to kill a cancerous cell if so detected by the present apparatus.

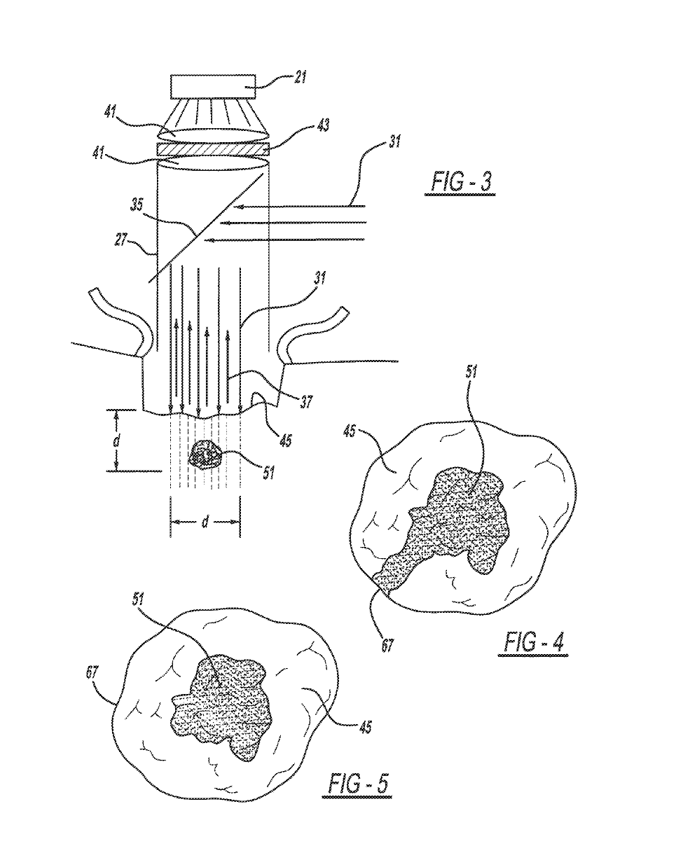

Referring to FIG. 4, a cancerous group of cells 51 will usually be exposed at a margin 67 of healthy tissue 45, thereby, allowing the fluorescing marking signal to be stronger and more easily found. With the present apparatus and method, however, the presence of even buried cancer cells 51 which are offset from margin 67 by distance d, as shown in FIGS. 4 and 5, can be quickly found with the present apparatus and method. Unlike conventional systems, the present apparatus does not require micron resolution (e.g., less than 100 microns). In fact, micron resolution would be disadvantageous with the present apparatus since a hand-held wand can be held with only an accuracy of 0.1 mm or greater. Therefore, the present apparatus advantageously provides a large field of view for the camera of between 10-100 mm², with a resolution of about 0.1 mm per pixel. The use of the preferred ultra-fast pulses allows the present apparatus to take advantage of the near-IR light that penetrates well into the tissue, and photon excitation which only occurs when it is excited by light that has not scattered. Essentially, the present detector and computer software are providing an automated “yes” or “no” determination report to the surgeon as to whether a cancerous cell is present at an unknown targeted location, rather than attempting to actually determining the location of the cancerous cell within the surrounding healthy cells. Alternately, a photodiode is used for detection in combination with a lock-in amplifier. This is a very sensitive and inexpensive way to detect fluorescence. In this mode the wand provides real-time yes/no diagnostic feedback without imaging. The wand itself can have a small light emitting diode that prompts the surgeon when a cancer cell is detected at the end of the wand.

In an alternate embodiment, a biomedical detection apparatus employs a red diode laser, having a wavelength between 560-650 nm, and uses a near-IR fluorophore, such as mPLUM, for the fluorescent chemical marker. Such a laser would allow the size of wand 27 to be significantly smaller (such as less than 10 cm long×1 cm diameter) while allowing the laser to be much less expensive. For this embodiment, it is desired to modulate the laser and detector in a matching coordinating manner in order to separate the reflected signal that is induced by the laser emission from background light. As another alternative, a photodiode or PMT single photon detector can be used instead of a CCD camera, however the camera approach advantageously provides targeting guidance to the surgeon through a video on the output display screen. Alternately, a photodiode is used for detection in combination with a lock-in amplifier.

When used for cancer detection during brain surgery, it is desired to attach wand 27 to an articulated or gantry robotic arm or the like. This will reduce inadvertent wand contact with the healthy brain tissue during rogue or unknown cancer cell detection after the known cancer cells are removed. Furthermore, the present apparatus and method can also be used for optical mammography through the skin of the patient. For mammography, however, greater laser power will be required, such as greater than 1 mJ, and using an average of 1,000 laser pulses per detection session. The present apparatus and method can also detect second or third harmonic generation so the computer controller and software automatically determine targeted bone density of the patient with the unfocused and ultrafast laser pulses.

B. Identification of Melanoma using Unfocused Chirped Pulses:

The present embodiment also applies to femtosecond lasers that are unfocused but employing alternating positively and negatively chirped laser pulses faster than 100 fs, and more preferably faster than 10 fs each, to create a contrast of cancer cells versus healthy tissue for sensing by a detector, without chemical markers. The goal is to focus on the systemic delivery of two-photon excitation without focusing to achieve two-photon excitation and other nonlinear optical processes over large volumes for medical diagnosis, identification and treatment purposes.

There have been some studies on the absorption and fluorescence characteristics of different types of melanin. For example, black hair contains eumelanin, whereas red hair contains a mixture of eumelanin and pheomelanin. The fractional content of eumelanin has been found to correlate with the likelihood that a mole is melanoma. Unfortunately, the absorption of all forms of melanin is extremely broad and featureless. The fluorescence signal is very weak and broad. Therefore, it is very difficult using linear spectroscopy to determine the ratio between eumelanin and pheomelanin. Warren S. Warren and John D. Simon had previously studied the transient absorption behavior of melanin; in particular, how spectroscopy changes as a function of delay between pump and probe pulses. They found important differences and concluded that nonlinear optical microscopy can distinguish different types of melanin. See, T. Matthews, et al., “Pump-Probe Imaging Differentiates Melanoma from Melanocytic Nevi,” Sci. Transl. Med., Vol. 3, Issue 71 (2011).

Nevertheless, pump probe techniques, such as those proposed by Warren, are cumbersome and time consuming. For each pump-probe delay time, one needs to average the results over a lengthy time period in order to obtain a good signal to noise ratio. When used for imaging as Warren has, for each pixel in the figure, the information needs to be obtained from at least two different time delays. For example, an image 100×100 pixels at ten different depths requires an analysis that could take from tens of minutes to an hour. Instead, the present invention uses an unfocused beam that compares the amount of emission observed for positive and negative chirped pulses. This way, in one second or less, the likelihood a nevus is melanoma is automatically determined by a controller without the need of precise imaging and inspection.

It is useful to have a fast (i.e., real-time) method to determine the ratio between eumelanin and pheomelanin to aid in the diagnosis of melanoma. This is accomplished by measuring the difference in light emitted/scattered from tissue irradiated with negatively and positively chirped pulses. The laser should be able to produce pulses centered at 800 nm with a pulse duration shorter than 20 fs and ideally 10 fs in duration, such that they have a sufficient intensity at both 750 and 820 nm. When those pulses are chirped by positive or negative 1000 fs², the pulses become longer in duration and there is a time delay between the 750 and 820 nm wavelength components of 100-200 fs. The 820 nm photons arrive earlier than the 750 nm photons for positively chirped pulses. The opposite occurs for negatively chirped pulses. By using the controller software to automatically alternate positively and negatively chirped pulses, a ratio of emitted light from the tissue is obtained that correlates with the fraction of eumelanin and pheomelanin. Furthermore, the difference observed by the detector for positive and negative chirp is maximized because eumelanin exhibits an excited state absorption that pheomelanin does not exhibit. That excited state absorption is maximized near a 300 fs delay after a bluer wavelength pump. This is why negatively chirped pulses will see less emission from eumelanin and more from pheomelanin. Conversely, positively chirped pulses will see less emission from pheomelanin and more from eumelanin.

Practically, alternating positive and negative chirp pulses are delivered at the in vivo tissue at a fast frequency. The frequency being faster than 10 Hz and ideally 1 kHz, and even better at MHz repetition rates. The slowest rates are achieved by a phase modulator while the faster rates can be achieved by splitting a negatively chirped pulse, delaying and chirping one portion and then recombining the pulses such that the first portion and the second portion are delayed by a time longer than one nanosecond. The delay should be sufficient that the detector is able to distinguish a signal that results from the first or second pulse. The amount of chirp is then adjusted so that one of the pulses is positively chirped while the second is negatively chirped. Thus, the signal obtained resolves differences between the two chirped pulses.

FIG. 7 illustrates how one negatively chirped pulse is split into two, one is unaffected (a) and the other one then goes through a dispersive material such as glass, and because of that, changes from negative chirp to positive chirp. Once the laser pulse is emitted from the laser, it is sent to a Mach-Zhender interferometer 71 that delays one optical arm with respect to the other. In one embodiment, the laser output is negatively chirped. In one of the arms of the interferometer, a long slab of glass of length 8 cm (but it can be between 1-10 cm) provides sufficient positive dispersion to counteract the negative chirp of the laser pulse and introduces a positive chirp of equal magnitude of that from the output pulses. The output of the Mach-Zhender interferometer is two pulses, one with negative and the other with positive chirp. The time delay between the two pulses is greater than one nanosecond. When used with an oscillator, the time delay is half of the repetition rate or about five nanoseconds. When used with an amplifier the time delay is just enough to be distinguished by detector 21 (see FIG. 2). Detector response time is typically two nanoseconds. Furthermore, the detector requires an optical filter to detect the desired signal, typically the fluorescence at a wavelength equal or longer than that of the probe. For non-imaging conditions, the detector itself is a simple photodiode, a biased photodiode, or it can be an avalanche photodiode or a photo multiplier.

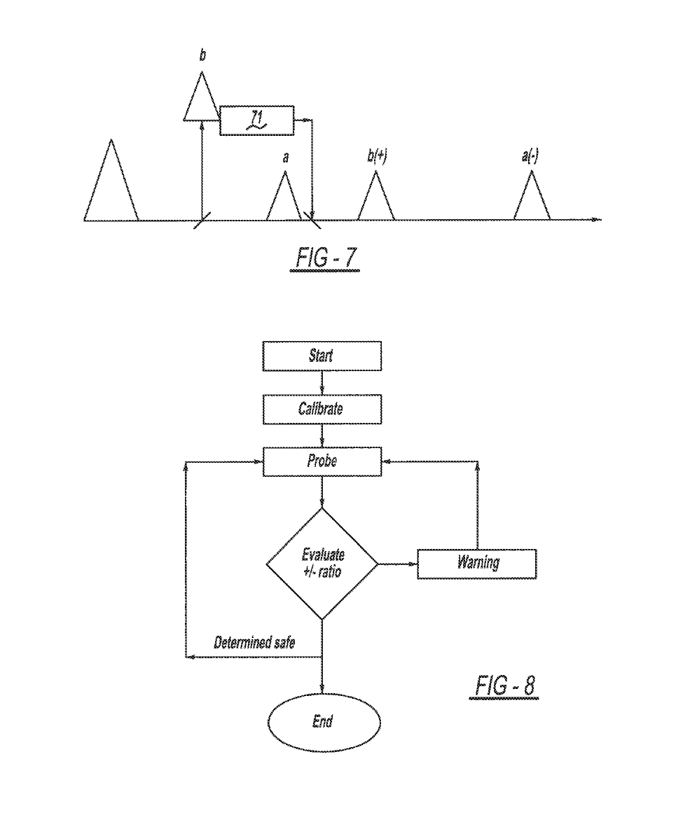

The system and software are calibrated by different hair samples and then it is ready for clinical use to diagnose melanoma, as shown in FIG. 8. With reference to FIG. 10, once an optical probe 73 and the system are calibrated, the doctor points the probe at a mole 75 on the patient's skin 77 and obtains a direct reading from the nearby computer display 47 (see FIG. 2) that indicates the likelihood that the mole is melanoma based on its detected and calculated ratio of eumelanin and pheomelanin.

Referring again to FIG. 8, the computer controller and software are calibrated for a given nonlinear optical spectroscopic change that is determined for two differently shaped pulses typically positive and negatively chirped pulses. In this case, the doctor is considering the determination of eumelanin and pheomelanin but it could be other chromophores such as the oxygenation of hemoglobin as probed in a small region based on differences obtained in the emitted light when irradiated by positive and negatively chirped pulses. When the probe is activated, the detector and controller measure the ratio and determine, based on stored values, if the ratio is “safe” or if the ratio is considered to indicate the presence of melanoma. If so, it displays a warning and provides the measured ratio (a number) to be interpreted by the doctor. The directness and speed of this method gives the doctor a greater degree of confidence. Moreover, the controller software accomplishes the calibration using standard calibration materials or the calibration can be done on the patient by probing healthy regions of skin. This provides a patient-specific calibration.

C. Treatment of Warts:

Also illustrated in FIG. 10, the unfocused femtosecond laser of the present invention can treat large volumes quickly for the treatment and killing of warts 75. This can be done with or without locally applied photodynamic therapeutic agents. For large warts, the top of the wart can be surgically cut before treating with the femtosecond laser. The surgeon thereafter activates the controller, and then holds the wand 73 against the wart on a patient 77 so it emits the ultrafast and unfocused laser beam pulses. No detection is required.

D. Treatment of Nail Fungus:

In Z. Manevitch et al., “Direct Antifungal Effect of Femtosecond Laser on Trichophyton Rubrum Onychomycosis, Photochemistry and Photobiology, 2010, 86: 476-479, it was determined that femtosecond laser pulses could be used to treat onychomycosis or nail fungi. This treatment was explored because the prior standard of care included ointments that do not penetrate the nail, photodynamic therapy that does not penetrate the nail, or systemic oral dose of antifungal medicines for long periods of time that can be toxic.

The method explored in the Monevitch publication, however, involved a titanium sapphire oscillator focused to a region that is limited to a few microns in x, y and z directions. The principal goal of the article was to determine the fluence at which the fungi were destroyed without causing damage to the nail. It was found that a laser fluence of 7×10³¹ photons m⁻² s⁻¹ was therapeutic but at 1.77×10³² photons m⁻² s⁻¹ the nail was damaged. This narrow range makes it difficult and too time consuming to treat the volume of a toe nail which is 100-1000 mm³.

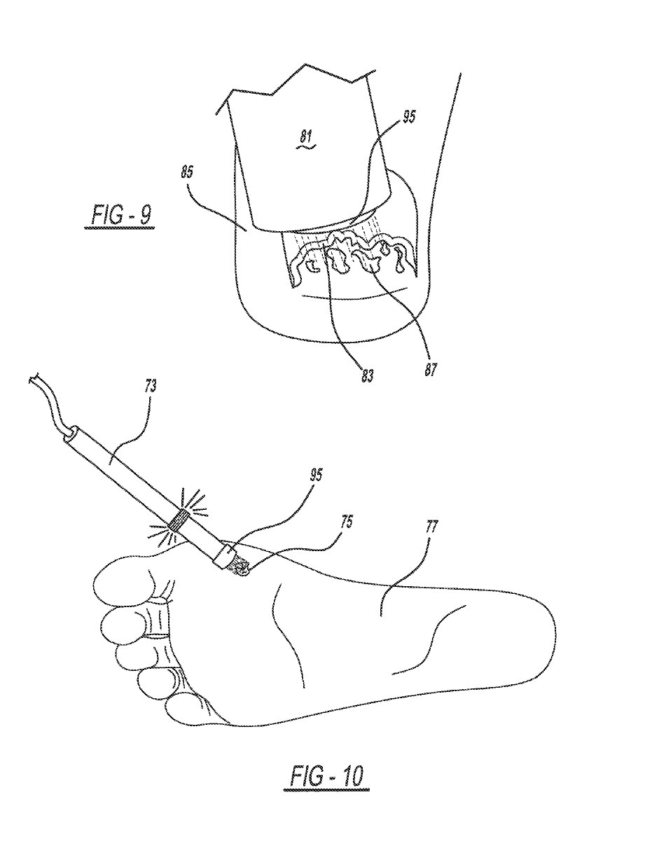

In contrast, the present invention embodiment of FIG. 9 shines amplified and unfocused femtosecond laser pulses through a handheld wand 81, thereby delivering the required fluence in less than ten seconds onto a toenail area 83 of a patient's toe 85 without scanning and without having to control the height of the laser with micron precision. The ultrafast laser pulses travel through nail 83 to reduce fungi 87 therebehind. This can also be used with fingernails. The femtosecond laser irradiation does not require a photodynamic therapeutic agent, to be effective. For severe cases, however, it can be combined with mild abrasion of the surface of the nail and also with a photodynamic therapeutic medication.

The wavelength of the pulses in the near infrared 700-1200 nm, and preferably 800 nm or 1050 nm. The peak fluence needs to be higher than 10⁹ W/cm² and lower than 10¹² W/cm². The pulse duration should be less than 100 fs and preferably as low as 10 fs. Furthermore, the energy density should be between 0.1-1.0 mJ/cm².

An amplified femtosecond laser capable of delivering the above energy densities is employed, preferably with a high power fiber oscillator. Minimum energy per pulse should be 10 micro-Joules, and the repetition rate is between 10 Hz to 100 MHz. It is likely that from an amplifier, it will be 10-1000 Hz. Delivery should be like the probe for fluorescence detection, but with no need for a fluorescence detector.

It is desireable to keep the laser irradiation contained because of the high energy. Thus, an optional O-ring or collar 95, such as a disposable silicon-rubber interface, is attached to the distal end of the laser wand. It is compressed against the tissue/nail and an interlock or switch is provided such that only when that interface is compressed can the laser be activated. Alternately, a capacitive sensor can activate an electrical circuit when it contacts the patient's skin.

Most virus, bacteria and fungi die with UV light. The laser activates two-photon excitation at a wavelength equivalent to half the wavelength of the incident light (for example, 400 nm for an 800 nm laser). These excitations release free radicals that are more likely to kill bacteria and fungi than human cells. Viruses are susceptible to genetic damage by UV light and become inactive by irradiation. Furthermore, topical laser-activated agent can be applied to enhance the action of the laser. These would be products that are good two-photon photodynamic therapy agents.

The virus in the wart gets genetically modified and becomes inactive. When a conventional laser is focused, its intensity varies with depth, achieving a maximum intensity at the focal point. Tight-focusing leads to a focal point that is microns in depth and impossible to control by a hand-held tool. In contrast, the unfocused laser of the present invention does not have such variations with depth. This makes it much easier to regulate the intensity and the volume of the tissue/nail being treated. Depth is limited in tissue by scattering to 1-2 mm.

E. Self-Contained Wands:

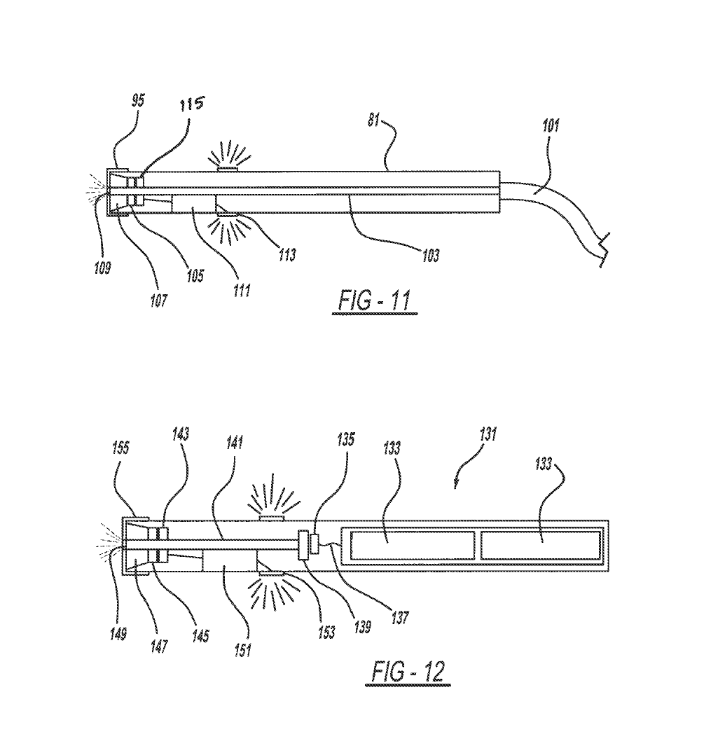

FIG. 11 shows a self-contained biomedical detection apparatus similar to that of the FIG. 9 embodiment. However, the present exemplary wand or housing 81 itself includes the detecter and controller. Output optical fiber 101 transmits laser light from a laser light source to a proximal end of rigid optical fiber 103 extending the length of wand 81. Ultrafast and unfocused laser pulses 109 are emitted through an aperature in a light shield or collar 95 at a distal end of wand 81, toward the patient tissue. The reflected or tissue emitted light is received by a light guide 107, filtered by a fluorescence filter 105 and sensed by a photodiode detector 115. The output signal from photodiode detector 115 is processed by software instructions stored in a microprocessor controller 111 which automatically determines if an undesireable cell, such as a cancer, is present in or on the tissue without imaging. If it is present, controller 111 will then activate a warning output, more specifically an indicator LED 113 mounted to and externally visible from wand 81.

Another self-contained variation can be observed in FIG. 12. In this embodiment of the biomedical detection apparatus, a wand 131 employs one or more electrical storage batteries 133. Batteries 133 are connected to and energize an excitation LED light source 135 via a wire 135 or other electrical circuit. The LED light passed through an excitation filter 139 and then along an elongated optical waveguide 141 from which it is emitted from an aperature 149 in a light shield or collar 155, toward the tissue of the patient. The fluorescent light emitted from the tissue is received by a fluorescence guide 147 in wand 131, and passes through a fluorescence filter 145. A programmed microprocessor controller 151 is connected to photodiode 143 for the real-time determination of whether a cancer cell is present or not within about 2 seconds. If so, it activates the warning indicator LED 153 or audio emitter to provide real-time notification to the surgeon.

An alternate construction uses multiple, different color LEDs 135 within wand 131. Microprocessor 151 automatically activates different combinations of color emissions from these LEDs onto the tissue. The detector and self-contained or remote microprocessor then cooperate to determine whether a specific type of cell is present or not the sensed fluorescence associated with the emitted color combinations.

It should be appreciated that these self-contained wands can be employed for any of the uses specified herein. Furthermore, specific features and hardware of any of the apparatuses discussed herein can be mixed and matched, and substituted with any of the others, although certain advantages may not be obtained.

While various embodiments of the present invention have been disclosed, it should be appreciated that other variations may also be employed. For example, different optical members may be provided for the laser and/or detector, however, the laser light emitted onto the tissue is unfocused. Furthermore, additional or reduced computer software instructions may be employed to achieve the same or similar functional results, although certain benefits may not be realized. Additionally, a binary Tr-step scanning pattern can be used with a phase mask SLM to cause automatic, computer controlled scanning of the tissue. Moreover, Raman scattering or CARS can be used for detecting cancer cells. For example, a Raman contrast agent with a vibrational frequency that is not common to living tissue, such as that from CN groups or from deuterated hydrocarbons, can be used as the contrast agent. The terms “doctor,” “surgeon” and “medical person” are used interchangeably throughout and are considered to be synonomous for this invention. It should also be appreciated that any of the features and devices described and shown for certain embodiments herein can be substituted, interchanged or added to any of the other embodiments, although many advantages may not be fully realized. It is intended that these and other variations fall within the scope of the present invention.

The invention claimed is:

1. A biomedical apparatus comprising:
unfocused laser or LED light emitted onto in vivo tissue;
a detector detecting if a certain light characteristic is received from an undesirable cell in the tissue; and
an electrical controller determining if the undesirable cell is detected, in less than five seconds from when the light is emitted onto the tissue.

2. The apparatus of claim 1, further comprising programmed software operating within a microprocessor of the electrical controller, the software further comprising:
first instructions comparing the sensed data from the sensor to stored data, in real time, and determining if the undesirable cell is detected in the targeted tissue; and
second instructions causing a warning to be transmitted to indicate if the undesirable cell is detected, during a surgery.

3. The apparatus of claim 1, wherein the light includes a red light emitting diode with wavelength between 560-650 nm, and the tissue has been injected with a chemical marker that includes a near-IR fluorophore.

4. The apparatus of claim 1, wherein the laser light is a set of laser beam pulses, each pulse having a duration less than 100 fs.

5. The apparatus of claim 1, wherein the photodetector is modulated at high frequency and the signal is amplified at the same frequency in order to isolate fluorescence induced by the light emitting diode and reject other sources of light.

6. The apparatus of claim 1, wherein the detector is modulated to match modulation of the laser light or light emitting diode light in order to separate a fluorescing signal from background light, and no interferometer or image mapping is used.

7. The apparatus of claim 1, wherein the detector is a photodiode mounted within a hand-held housing which emits the light, and the electronic controller being mounted to the housing.

8. The apparatus of claim 1, further comprising:
a hand-held wand, an optical fiber coupled to the wand, the detector mounted on the wand and a beam splitter located in the wand; and
a surgeon holding the wand to aim the laser light, passing through the beam splitter, to be targeted at the tissue;
the beam splitter reflecting any light fluorescing from the undesirable cell to the detector.

9. The apparatus of claim 1, further comprising a chemical marker causing the undesirable cell, which is a cancer cell, to fluoresce a color different from adjacent healthy tissue.

10. The apparatus of claim 1, wherein the electrical controller operably determines if the undesirable cell is present within at least a 10 mm³ volume of the tissue inside of a surgically open incision in the patient within which the laser light is emitted.

11. The apparatus of claim 1, wherein the electrical controller determines and reports whether the undesirable cell, which is a cancer cell, is present or not at a targeted location without determining where the cancer cell is actually located.

12. The apparatus of claim 1, wherein alternating positive and negative chirp are used to assist in the determination by the controller.

13. (canceled)

14. A biomedical apparatus for use on a living patient, the apparatus comprising:
a laser emitting unfocused laser light on exposed internal tissue of the living patient;
a chemical marker located in the tissue;
a sensor operably receiving a signal from the marker if activated by the laser light; and
an electrical controller determining if a cancer cell is present in the tissue based on sensed data from the sensor in less than two seconds from when the laser light is emitted onto the tissue being targeted.

15. The apparatus of claim 14, wherein the laser light is a set of laser beam pulses, each pulse having a duration less than 10 fs and with a peak intensity of at least 10¹⁰W.

16. The apparatus of claim 14, wherein the sensor is modulated to match modulation of the laser light in order to separate a fluorescing signal from background light, and no interferometer or image mapping is used.

17. The apparatus of claim 14, further comprising:
a hand-held wand, a pulsed laser coupled to the wand by an optical fiber, the sensor mounted on the wand and an optic located in the wand to separate excitation light from fluorescence; and
a surgeon holding the wand to aim the laser light to be targeted at the tissue;
the optic transmitting from targeted tissue to the detector.

18. (canceled)

19. The apparatus of claim 14, wherein the electrical controller operably determines if the cancer cell is present within a 10 mm³ volume of the tissue inside of a surgically open incision in the patient within which the laser light is emitted.

20. The apparatus of claim 14, wherein the sensor is a photodiode mounted within the hand-held wand which emits the light, and the electronic controller being portable with the wand.

21-37. (canceled)

38. A biomedical apparatus comprising:
a hand-held wand delivering unfocused monochromatic light through an excitation filter onto in vivo tissue;
a detection filter rejecting light used for excitation;
a detector detecting if a certain light characteristic is received from an undesirable cell in the tissue; and
an electrical controller determining if the undesirable cell is detected, in substantially real-time from when the light is emitted onto the tissue.

39-41. (canceled)

42. The apparatus of claim 38, wherein the photodetector is modulated at high frequency and the signal is amplified at the same frequency in order to isolate fluorescence induced by the light emitting diode and reject other sources of light.

43-79. (canceled)
